(19) United States
(12) Patent Application Publication
(10) Pub. No.: US 2006/0188492 A1
(43) Pub. Date: Aug. 24, 2006

(54) Topical management of ocular and periocular conditions

(75) Inventors: Kenneth T. Richardson, Anchorage, AK (US); Don C. Pearson, Lakewood, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ChronoRX LLC, an Alaska Limited Liability Company, Anchorage, AK (US)

(21) Appl. No.: 11/332,625

(22) Filed: Jan. 13, 2006

Related U.S. Application Data
(60) Provisional application No. 60/644,070, filed on Jan. 13, 2005.

(51) Int. Cl. A61K 38/05; A61K 38/43; A61K 31/525; A61K 31/56; A61K 31/385

(52) U.S. Cl. 424/94.1; 514/18; 514/171; 514/250; 514/440

(57) ABSTRACT

Chronic glaucoma, cataract, ocular and periocular aging are treated and prevented by the administration of agents that affect metabolic subsystems such as (i) mitochondrial bioenergetics, (ii) free radical moderation and glutathione maintenance, (iii) constitutive nitric oxide/endothelin-1 balance, and (iv) calcium wave signaling and associated neuronal excito-toxicity. Included among the agents are tetrahydrobiopterin, R-alpha-lipoic acid, coenzyme Q10, 17 alpha-estradiol, and glutathione.

### CROSS REFERENCE TO RELATED APPLICATION

This application claims benefit from U.S. Provisional Patent Application No. 60/644,070, filed Jan. 13, 2005, the contents of which are incorporated herein by reference in their entirety.

### BACKGROUND OF THE INVENTION

1. Field of the Invention

This invention resides in the fields of pharmacology and biochemistry. It relates to the topical ocular application of defined dosage forms as methods of use for the treatment of undesirable ocular and periocular conditions. It describes therapeutic uses of individual or complexed physiological molecules applied to the surface of the eye as modifiers of cell physiology at the plasma cell membrane and of subcellular organelles, including mitochondria, within the anterior and posterior segment of the eye and the periocular tissues.

2. Description of the Prior Art

A biological system consists of a definable set of metabolic nodes and a web of interactions between these nodes—i.e., it is a metabolic response network that contains multiple subsystems. The invention defines those metabolic subsystems, which when disturbed, individually or collectively are involved in causing the ocular and periocular pathologies targeted by the invention. The metabolic subsystems addressed by the invention are: 1) mitochondrial bioenergetics, 2) free radical modulation, 3) the balance between constitutive nitric oxide/endothelin-1 levels, and 4) the cellular control of calcium wave signaling.

The conditions addressed by the invention, which occur when some or all of these metabolic subsystems are disturbed, are: 1) chronic glaucoma, 2) cataract, and 3) ocular and periocular aging. Since these pathophysiologies share etiologic disturbances of some or all of the metabolic subsystems, it is not surprising that a variety of these conditions may coexist when one (or certainly when all) of the metabolic subsystems become dysfunctional.

The invention reduces intraocular molecular pollution; that is, the excessive production of otherwise important physiological molecules, like free radicals and endothelin-1, when these arise from dysfunctional vascular endothelium of the ciliary body and retina. In some pathological conditions, and in ordinary aging, the ciliary body and/or retina become a ‘smokestack industry’ within the eye. The invention favorably alters the dysfunctional metabolic subsystems involved, and the diseases and conditions caused or aggravated by this intraocular pollution.

Therapeutic uses are described for the invention in the clinical management of chronic glaucoma, cataract, ocular and periocular aging.

The complexity of these areas of metabolic subsystem dysfunction and their interdependence demands the administration of multiple orchestrated components to prevent or ameliorate the targeted conditions. A single therapeutic “silver bullet” is unrealistic when dealing with the complexity of these conditions. The invention addresses this complex etiologic interdependence with multicomponent, orchestrated formulations based upon established and rational science, not myth-riddled randomness.

### DESCRIPTION OF THE INVENTION

Because of the existence of common pathways for some biochemical and physiological processes, the recurrence of some molecules of the invention and the repetition of other unique variables that are disease specific, this document is organized as follows to avoid redundancy and yet permit development of inherent complementarities and variations:
I. Metabolic Subsystems—modifiable by interrelated groups of active ingredients and processes that are globally applicable to each of the clinical targets.
   A. Mitochondrial Bioenergetics
   B. Free Radical Moderation (& Glutathione Maintenance)
   C. Constitutive Nitric Oxide/Endothelin-1 Balance
   D. Calcium Wave Signaling (& Associated Neuronal Excito-toxicity)
II. Clinical Targets—pertinent biochemistry of the primary clinical intraocular ophthalmic targets of the invention and the influence upon each by the invention.
   A. Chronic Glaucoma
   B. Cataract
   C. Ocular and Periocular Aging

The invention describes very specific formulations consisting of therapeutically effective, complementary or synergistic molecules that act as active ingredients at the cell membrane level of cellular physiology, to enhance or preserve cell membrane stability and efficiency (in particular at the caveolae), that function to improve cellular respiratory bioenergetics at the mitochondrial level and help to maintain the functionality of other subcellular organelles.

The molecules in the formulations described for the Clinical Targets of Section II are chosen more specifically to address identifiable nodes of physiological instability or potential failure within metabolic subsystems that are of particular importance as individual risk factors for an individual target, and are frequently different for each of the clinical targets.

It should be kept in mind that in this therapeutic arena, errors of commission are as important as errors of omission and each formulation contemplated by the invention has been designed as a freestanding, functional unit. These formulations should not be seen as mere collections of physiological molecules randomly brought together in all encompassing lists of everything available at the molecular supermarket. The “More is Better” approach is specifically avoided.

### I. Metabolic Subsystems

Shared Physiology

Within the eye the most metabolic active tissues are the ciliary body and the retina. Furthermore, the ciliary body contains an inducible and very active monooxygenase system prone to the generation of oxidizing free radicals (FR). These FR, combined with those produced via the cyclooxygenase pathway, can result in ocular damage. In the retina the photoreceptor rhodopsin is itself a photodynamic agent that initiates significant FR formation. Additionally, concentrations of retinal polyunsaturated fatty acids (PUFAs) in the photoreceptor membranes form significant FR by auto-oxidation. As a result, the ciliary body and retina are the greatest ocular source of pathological levels of production of intraocular FR and endothelin-1 (ET-1).

An aging or dysfunctional ciliary body becomes the ‘smokestack’ of the anterior segment of the eye, pouring excess FR and ET-1 into the aqueous humor, damaging the lens of the eye (cataract), which is bathed in aqueous, and the trabecular meshwork (chronic glaucoma), which is the drain through which the aqueous humor must exit the eye.

If a dysfunctional ciliary body is the ‘smokestack’ of the anterior eye, the aging or unhealthy retina certainly qualifies for the same distinction in the posterior segment of the eye. For example: The retina can produce excessive FR and ET-1, which are an established risk factor in the optic nerve damage associated with chronic glaucoma.

The invention reduces the production of these by all ocular tissues, including the ciliary body and the retina, and also reduces the adverse effects of them (and of homocysteine) on exposed cells. The invention thereby favorably alters the development of chronic glaucoma, cataract, ocular and periocular aging

Free Radicals (FR)

FR are physiologically important. They are critical for normal cell function, but if produced in excess, they damage cell membranes and subcellular organelles and can trigger a cell death cascade. Pathological levels of FR occur in a variety of circumstances including aging. As they age, mitochondria become less efficient in energy production. As a result, the percentage of the superoxide anion (O^{•}) produced during mitochondrial respiration increases progressively from the “physiological 5% in youth” to higher levels. These elevated levels challenge and may overcome the normal antioxidant buffering capacity within the mitochondria; in a worst case, this leads to bioenergetic collapse and premature cell death (apoptosis). The situation is exaggerated when a deficiency of antioxidants exists. This is especially true if the deficiency is (or results in) inadequate levels of mitochondrial glutathione.

The main oxidizing FR are oxygen-derived metabolites (e.g., superoxide anion (O^{•}), hydrogen peroxide (H₂O₂), hydroxyl radical (OH⁻), hypochlorous acid (HOCl), chloramines (NH₂Cl), nitrogen oxides (NO^{•}), ozone (O₃) and lipid peroxides). Living organisms produce them continually, either in the intracellular compartment by the mitochondrial respiratory chain and mixed function oxidase system, or in the extracellular compartment, especially by phagocytes. The body possesses complex protective antioxidant systems against this potentially toxic production of FR, such as superoxide dismutase, catalases, metallic ion sequestration, enzymes which degrade proteins damaged by FR, metabolizing hydroperoxides, DNA repair processes, antioxidant vitamins E, C and, in particular, glutathione and the glutathione enzyme system. Of exceptional interest is the interplay between mitochondrial bioenergetics, oxidative stress and glutathione. Under normal conditions a physiological steady state is established between the production of oxidants and their neutralization by antioxidants. The mitochondrial respiratory chain (about 95% efficient when functioning normally) results in the production of ATP (energy), CO₂ and H₂O. Even under these normal conditions about 5% of the oxygen ends up as FR (initially, superoxide; then superoxide→H₂O₂; then H₂O₂→OH^{•}). OH^{•} is the worst, most toxic of the FR. With aging, mitochondria become less efficient in their use of oxygen, and there is a progressive increase in the superoxide→OH^{•} process, which in turn increases aging. The invention is designed to moderate this feed-forward pathological phenomenon.

The invention maintains maximum efficiency of oxygen usage by the mitochondria of the ciliary body and retina (as well as other ocular and periocular tissues), reducing the production of pathological levels FR. In addition, it counteracts the FR that have been produced. It not only maintains physiological levels of constituent endothelial nitric oxide (cNO), but also reduces increases of ET-1 induced by FR; by so doing it maintains physiological cNO/ET-1 balance. Furthermore, in some of its configurations the invention reduces homocysteine, which is also correlated with excessive ET-1. The latter, an amino acid peptide, is a powerful vasoconstrictive agent, which causes a further reduction of mitochondrial respiration, adversely alters mitochondrial gene expression and can act as an excitotoxic NMDA (N-methyl D-aspartate) receptor ligand in the retina.

Endothelin-1

Physiological levels of ET-1 are of importance for vasomotor stability, but when produced in excess from dysfunctional or aged vascular endothelium of the ciliary body or retina it leads to various pathologies. Excess ET-1 occurs routinely during aging as endothelial cell membranes are damaged by cumulative pathological amounts of free radicals, intracellular calcium, extracellular insulin or glucose, and homocysteine. As a result, aging reduces the efficiency of the multifactorial, complex biologically defense system that maintains the critical physiological function of the ciliary body and retina. Applied topically to the surface of the eye and conjunctiva, the invention will penetrate the eye and periocular tissues and favorably modify this important biological network at multiple nodes.

The examples that follow illustrate the following Metabolic Subsystems that are favorably modulated by the invention:
A. Mitochondrial Bioenergetics
B. Free Radical Moderation (& Glutathione Maintenance)
C. cNO/ET-1 Balance
D. Calcium Wave Signaling (& Associated Neuronal Excito-toxicity)

A. Mitochondrial Bioenergetics

Loss of Bioenergetic Efficiency

Bioenergetic collapse spells cell death. In the anterior segment of the eye an unfortunately frequent example is cellular death in the trabecular meshwork leading to elevated intraocular pressure and progressive glaucoma. In the posterior segment of the eye, death of the retinal ganglion and glial cells of the optic nerve, secondary to this elevated pressure, results in visual loss progressing to glaucoma blindness. In the lacrimal gland, acinar cell death reduces the quantity and quality of tears, often leading to severe symptoms associated with a “dry eye”.

Prevention of mitochondrial dysfunction is one goal of the invention.

The structural and pathophysiological results of mitochondrial dysfunction include: 1) loss of the mitochondrial inner transmembrane potential, 2) uncoupling of the respiratory chain, 3) hyperproduction of superoxide anions, 4) outflow (loss) of matrix calcium, 5) outflow (loss) of matrix glutathione, and 6) release of soluble intermembrane proteins. This eventually concludes in a bioenergetic catastrophe associated with necrosis of the plasma membrane and/or the presence of apoptogenic caspase proteases; the latter activated by mitochondrial proteins that leak into the cytosol (e.g., cytochrome c, apoptosis-inducing factor et al) and by secondary endonuclease activation. The relative rate of these two processes (bioenergetic catastrophic necrosis, or protease and endonucleases induced apoptosis) determines whether cell necrosis or apoptosis will ultimately prevail. (The method of cell death is important in relation to the health of adjacent cells since necrosis is associated with inflammation that can further stress cells in the locale . . . another feed-forward pathological mechanism that is addressed by the invention.)

Caveola-like mitochondrial membrane domains globally mediate apoptosis. The mitochondrial permeability transition pore (mPTP), also called the mitochondrial megachannel, is a multiprotein complex formed at the contact site between the mitochondrial inner and outer membranes, exactly the same location at which the apoptosis-inhibiting oncogenes Bax, Bcl-2 and Bcl-XL are particularly abundant.

The mPTP are in part regulated by proteins from the Bcl-2 family. These “Bcl-2” pores resemble islands floating, iceberg-style in a sea of sphingomyelin. When converted to ceramide, sphingomyelin creates an apoptotic (opening) signal to the mPT pore. The mPTP is importantly involved with regulating matrix Ca²⁺, cellular pH and modulating transmembrane potentials. It functions as a gated ion channel with little, if any, ion selectivity. Pathological levels of Ca²⁺ and/or mitochondrial oxygen radicals lead to much higher permeability at the mPTP. This irreversible opening of mPTP releases additional apoptogenic factors that activate cysteine-aspartate proteases (caspases) and endonucleases. And as mentioned above, these are the principal orchestrators of apoptosis.

In addition, mitochondrial respiration can trigger a further apoptosis by a ceramide-signaled release of cytochrome c from the mitochondrial membrane into the cytoplasm. The cytochrome c release also activates the caspase cascade, but this time via interaction with a cytosolic protein called APAF-1. In contrast to the mechanisms, which operate after a pathological mPTP opening (see above), cytochrome c can be released from “energized” (normal polarized state) mitochondria; that is, before the inner membrane has lost its integrity and the mPTP is irreversibly opened.

Adenine nucleotide translocase (ANT) is a major protein component of the inner membrane portion of the mPTP complex. It is responsible for exporting ATP into the cytoplasm in exchange for adenosine diphosphate (ADP). It is a dynamic, “flipping” gated pore, the frequency of the flips (open pore vs. closed pore) being determined by the Ca²⁺ concentration of the mitochondria. ANT therefore exists in one of two states: the c-state, wherein its substrate-binding site faces the cytoplasm (open state); and the m-state, wherein the binding site faces the mitochondrial matrix. Ca²⁺ allosterically governs its open-close frequency. Although mitochondria are immune to rapid cytosolic Ca²⁺ transients, they undergo an apoptotic permeability transition if cytosolic Ca²⁺ is maintained above a critical limit. And while Ca²⁺ determines the flicker frequency, cytosolic ADP is the catalytic switch; that is, no conformational change of ANT will occur unless there is ADP “on board” to be imported into the mitochondrion. This implies that the m-state of ANT is its native state.

The outer mitochondrial membrane at the mPTP complex contains a non-selective voltage-dependent anion channel (VDAC), which allows metabolite and ion access to the solute-specific transport systems of the inner membrane (such as ANT), which are complexed with the VDAC. VDAC bind to cytosolic kinases, most notably hexokinase and creatine kinase. The VDAC-ANT complex is further complexed with cyclophilin-P (CyP-D) of the mitochondrial matrix. CyP-D is a water-soluble protein that catalyses the folding of newly imported proteins, and also acts as a binding site for mitochondrial matrix Ca²⁺. The entire VDAC-ANT-CyP-D complex undergoes a fundamental conformational change in the presence of matrix free Ca²⁺ that allows it briefly to flicker into an open pore state.

As mitochondrial Ca²⁺ increases so does the abundance of contact sites between inner and outer mitochondrial membranes. When Ca²⁺ reaches a critical limit the adenine nucleotide translocase (ANT) is converted into a non-specific open pore. This conversion requires the binding of Ca²⁺ to CyP-D. Importantly, this binding is increased in response to oxyradicals (very notably in reperfusion following ischemia). A mitochondrial permeability transition (MPT) is the technical term given to the opening of non-specific pores in the inner membrane of mitochondria with an outflow of matrix Ca²⁺ and glutathione, and the release of soluble intermembrane proteins, which activate caspases and endonucleases (see above for the negative effects of this activation). Additionally, this outflow of matrix Ca²⁺ during this transition state activates calpain, one more enzyme involved in apoptogenesis. (Although calpain is a normal, intracellular cytosolic cysteine protease, when activated it leads to the degradation of cytoskeletal proteins, becoming an integral part of the apoptotic process, complementing the caspases.)

In summary: Via these various routes, most of which are Ca²⁺ modulated, mitochondria are converted from organelles whose production of ATP sustains the cell, to instruments of apoptosis and/or necrosis.

The ciliary body and retina have particularly high-energy requirements making each especially prone to inducing high levels of mitochondrial respiratory byproducts. After cellular stresses the mitochondria are often the victims of oxidative injury leading to necrotic and/or apoptotic cell death. The mitochondrial permeability transition preceding cell death does not occur uniformly during apoptosis. Initially, a small proportion of mitochondria undergo permeability transition. This increases to nearly 100% over time. Eventually the accumulated inhibition of oxidative phosphorylation progresses to a level where the opening of high conductance permeability transition pores in the mitochondrial inner membrane abruptly increases the non-selective permeability of this membrane to solutes. Under these circumstances, NAD(P)H oxidation, increased mitochondrial Ca²⁺ and mitochondrial generation of oxyradicals contribute further to the permeability transition. As a result, ciliary and retinal cellular apoptosis and necrosis may begin with a common stress or death signal and progressing by shared pathways, ultimately conclude in either cytolysis (necrosis) or programmed cell death and resorption (apoptosis) depending on many factors, but particularly upon the availability of ATP and Ca²⁺ flows.

These moves along the slippery slope from bioenergetic collapse to premature apoptosis are moderated by the invention.

Components of the invention (including their precursors, derivatives homologs, and analogs) that have a primary modulating effect on mitochondrial bioenergetics are as follows, one or more of which may be included in a product formulation of the invention:
1) Coenzyme Q10 (and homologues)
2) L-carnitine
3) 17 α-estradiol

Preferred

| | |
|---|---|
| Coenzyme Q10 | 0.003% to 2.250% |
| L-carnitine | 0.008% to 2.695% |
| 17 α-estradiol | 0.008% to 2.962% |

Cells can metabolize a variety of organic molecules to produce ATP within their mitochondria. The invention specifically includes 17 α-estradiol, coenzyme Q10, and L-carnitine that are involved in mitochondrial ATP production and are therefore important for stabilizing mitochondrial bioenergetics. Some of these elements are reduced or nutritionally lacking in a variety of clinical states, or are known to be present in insufficient quantities with aging. The cytoprotective and mitoprotective properties of 17 α-estradiol (an isomer of estrogen without its hormonal effect) prevents cell death in large measure by maintaining a functionally intact mitochondrion, becoming intercalated into the mitochondrial inner membrane (as well as the cell plasma membrane), stabilizing it and contributing to maintaining its physiologically critical selective permeability.

The fuels for ATP production: glucose, fatty acids and amino acids are transported into the cell. Various transporters located in the inner cellular membrane facilitate the exchange of molecules between the cytoplasm and the mitochondrial matrix.

Glucose is transported into the cell under the influence of insulin. Once in the cytoplasm, glucose is activated by phosphorylation and cleaved into two pyruvate molecules. During this anaerobic, glycolytic process 2 ATP molecules are formed, which represents just a small fraction of the chemical energy stored in glucose. Pyruvate molecules are then transported into the mitochondrial matrix by a pyruvate specific transporter, driven by a proton gradient generated by the mitochondrial electron transport chain.

The reactions involving pyruvate in the mitochondrial matrix (Krebs cycle) produce 2 ATP molecules, 8 NADH molecules and 2 FADH₂ molecules. NADH donates electrons to a mitochondrial electron transport (respiratory) chain resulting in the production of 32 to 34 molecules of ATP. The chain consists of three protein complexes integrated in the inner mitochondrial membrane and two mobile carrier molecules, CoQ10 and cytochrome c.

This respiratory chain is a part of the process of oxidative phosphorylation that catalyzes the transport of electrons from the reduced form of nicotinamide adenine dinucleotide (NADH) via CoQ10 to molecular oxygen. Most of the energy released is used for the formation of an electrochemical (hydrogen ion) gradient across the inner mitochondrial membrane, which ultimately drives the synthesis of ATP by mitochondrial inner membrane bound ATP synthase.

Various pathways feed electrons into the chain. The carrier molecule, NADH (synthesized from nicotinamide) captures energy by accepting high-energy electrons. At complex I it transfers electrons to CoQ10 via flavin mononucleotide (FMN) and flavin adenine dinucleotide (FAD), for which riboflavin is an essential precursor. (FAD is also a cofactor for glutathione reductase a critical enzyme indirectly involved with limiting FR damage in the mitochondria.) The energy from of these high-energy electrons is circuitously directed to the energy storage molecule, ATP. At the conclusion of this oxidative phosphorylation process oxygen is the necessary final electron acceptor, which allows pyruvate to be fully broken down and its energy harvested as ATP. Therefore, during the respiratory process, hydrogen ions are pumped from the mitochondrial matrix into the mitochondrial intermembrane space, creating a hydrogen ion gradient across the inner mitochondrial membrane. These hydrogen ions then move back into the mitochondrial matrix through specific protein channels, which are activated to produce ATP. ATP diffuses from the mitochondria into the cytoplasm, providing stored energy necessary for cellular metabolism.

CoQ10 is an essential cofactor of the mitochondrial electron transport chain. CoQ10 (CoQ10) transfers electrons from complexes I and II of the mitochondrial respiratory chain to complex III. In turn, complex III delivers them to the small protein, cytochrome c. Cytochrome c then passes on electrons to complex IV (cytochrome c oxidase), which catalyzes the final transfer of the electrons to oxygen. CoQ10 also serves as a potent mitochondrial antioxidant directly and indirectly by rejuvenating α-tocopherol.

The cellular metabolism of fatty acids requires the cytosolic carnitine cycle. Carnitine is central to the translocation, via a specific transporter, of the long chain acyl-CoA across the inner mitochondrial membrane. Acyl groups of activated fatty acids are esterified with carnitine in the cytoplasm. The acylcarnitine formed is then transported into the matrix space by a carnitine transporter in exchange for free carnitine. Once in the matrix the acyl residues are transferred back to CoA.

Oxygen in the mitochondrion is the electron acceptor at the end of the respiratory chain with a resultant formation CO₂ and H₂O.

B. Free Radical Moderation (and Glutathione Maintenance)

These components of the invention (including precursors, derivatives, homologs and analogs) have a primary effect on free radical moderation and GSH maintenance. Examples are the following, one or more or which may be included in a product formulation of the invention:
1) Glutathione or its monoethyl ester (and including the precursors cysteine, oxothiazolidine, mercaptopropionylglycine and N-acetylcysteine; and analogs)
2) α-Lipoic acid (all forms, including R-D-α-lipoic acid and the racemic form, thioctic acid)
3) N-acetylcysteine

Preferred

| | |
|---|---|
| Glutathione | 0.003% to 2.383% |
| R-α-lipoic acid | 0.002% to 3.150% |
| N-acetylcysteine | 0.008% to 2.944% |

Most cellular oxygen free radicals are generated within the mitochondria. For cellular health it is imperative that a coordinated antioxidant defense is consistently available within mitochondria to protect mitochondrial DNA (the latter accumulates genomic damage during aging) and mitochondrial membranes from FR damage, and to limit FR escape from the mitochondria into the cytoplasm, with subsequent damage to nuclear DNA, the plasma membrane and an ultimate escape from the cell. As mentioned, the efficiency of the mitochondrial electron transport chain progressively decreases with age: It is 95% efficient in oxygen utilization in youth with 5% of the oxygen ending up as free radicals. During the aging process efficiency gradually declines and oxygen free radicals are produced at a progressively greater rate. It is understandable that diseases which are directly or indirectly related to FR, such as chronic glaucoma, cataract and other age-related processes of the anterior segment of the eye are more likely to occur and progress more rapidly as a person ages. The FR that escape from the ciliary body into the aqueous humor are swept over the surface of the lens into the anterior chamber, damaging tissue along the way, to end in a final slow and concentrated passage through the trabecular meshwork.

Those FR that diffuse from the aqueous into the posterior ocular segment are added to those escaping from the retina; a possible contributing factor of optic nerve damage in glaucoma.

Within the mitochondria the most important antioxidant is glutathione (GSH), functioning in a cellular defense cascade with α-tocopherol, ascorbate and lipoate. GSH is synthesized within the cell (see below). GSH, a tripeptide, penetrates cell membranes less well than its precursors, such as cysteine, oxothiazolidine, mercaptopropionylglycine and N-acetylcysteine. These are contemplated by the invention to ensure the maintenance of normal or youthful rates of cellular GSH synthesis.

α-Tocopherol, ascorbate and lipoate are all important because they conserve GSH by neutralizing FR, thus lessening its (GSH) workload. As an antioxidative team they effectively work to conserve and rejuvenate each other thereby maintaining their appropriate redox state. Because of its lipid solubility α-tocopherol works well within mitochondrial and plasma membranes. Water-soluble ascorbate compliments this by functioning within the mitochondrial matrix, mitochondrial intermembrane space and the cytoplasm.

Lipoic acid (LA) is a very effective mitochondrial antioxidant that is particularly useful in maintaining the level of the GSH within the mitochondrion (see below). There is even evidence that LA may act as an intra-mitochondrial precursor of GSH; since LA is soluble in both lipid and water it can augment the antioxidant effect of ascorbate within the matrix and α-tocopherol within the membrane.

GSH (γ,glutamyl-cysteinyl-glycine) is produced intracellularly from the amino acids glutamic acid, cysteine and glycine. GSH is the major low molecular weight thiol compound of the living animal cell. In addition to actions as a direct antioxidant, GSH acts as a cofactor for the protective enzyme selenium-dependent glutathione peroxidase (GSHPx). Zinc (Zn²⁺) is a necessary trace element in GSH synthesis. Tissue levels of GSH and GSHPx decline with aging. This is especially notable in the lens of the eye, where this decline of GSH is associated with cataract development and in chronic glaucoma. The important global protective role of GSH in aging is firmly established. GSH directly increases aqueous outflow through the trabecular meshwork of the eye, thereby demonstrating the potential for lowering intraocular pressure and indirectly, by metabolizing and detoxifying the hydrogen peroxide, which is secreted into the aqueous humor by the ciliary body and which modifies the glycosaminoglycan secretory patterns of the cells of the trabecular meshwork leading to the dysfunctional intraocular pressure control that occurs in aging eyes with chronic glaucoma. It should be kept in mind that chronic glaucoma is most common after age 50 years, as is cataract.

Cell death (apoptosis) can be initiated by excess FR resulting from inadequate mitochondrial GSH with subsequent loss of mitochondrial membrane potential, inner membrane structural damage, and mitochondrial condensation. These changes lead to cytochrome c release from the mitochondrion into the cytoplasm and activation of the caspase apoptotic cascade. This has been extensively discussed above.

Tumor necrosis factor-α (TNF-α) induced apoptosis is also dependent upon GSH levels. In cells with decreased levels of the GSH, TNF-α by itself may act as an apoptotic agent. Fortunately, the antioxidant α-lipoic acid (see, below), which protects against the loss of GSH in cells exposed to TNF-α, completely prevents mitochondrial damage, caspase activation, cytochrome c release, etc., and therefore inhibits both mitochondrial and cytokine signaled apoptosis. Because cellular injury is regulated to a large extent by the GSH content and availability, it is apparent that apoptosis involves both oxidative injury and mitochondrial damage.

Normalization of the GSH level exerts a neuroprotective effect, of pivotal importance for the optic nerve and for trabecular neurons in the face of clinical chronic glaucoma. Human GSH levels cannot be raised by direct supplemental administration in the diet. Because of the limited absorption of the GSH tripeptide, other physiological molecules capable of preserving reduced GSH and supporting the production of GSH, and which are efficiently absorbed, have been necessary when oral supplements were contemplated. The topical application of these molecules, including GSH, directly to an absorbing surface (in this case the eye and conjunctiva) provides a more efficient method of delivery since it has no reliance upon multiple intestinal inefficiencies and has less reliance upon peripheral vascular system stability to provide appropriate accumulations in the targeted, ophthalmic tissues. Additionally, the conjunctival membrane pores (for passage of hydrophilic molecules) are about twice the size of those in average plasma membranes (which limit passage of hydrophilic molecules with a molecular weight above 400), therefore the conjunctiva and underlying sclera with even a much larger pore size will accommodate passage of the peptide glutathione (molecular weight 307).

α-lipoic acid (LA) is a physiological substance with considerable therapeutic value and low toxicity that is uniquely complementary with the other components of the Free Radical Moderation Group of the invention and very important in preventing and ameliorating the ocular pathologies of both the anterior and posterior of the eye described in the invention. LA (1,2-dithiolane-3-pentanoic acid) with a chiral center at the C3 carbon atom is the R (natural) configuration. (The S configuration of LA, the racemic mixture, and β-lipoic acid are biologically less potent, particularly in the mitochondrion.)

LA is a dithiol; its reduced form, dihydrolipoate is a disulfide reducer with a low redox potential. This, plus its activity as a radical scavenger (especially the hydroxyl radical), restores membrane fluidity resulting from oxidative stress. LA in its natural state tends to be protein bound by an amide linkage to a lysine. However, after administration significant levels of the unbound form occurs and the therapeutic roles of free lipoate increase. Usefully, both lipoic acid and its reduced form dihydrolipoic acid act as antioxidants. In fact, it has been shown that dihydrolipoic acid is able to exert a curative effect on oxidative-stress induced diseases by reversing the oxidative damage of macromolecules, i.e., not just preventing oxidative-stress induced damage.

Redox-sensitive mechanisms are involved in undesirable vascular smooth muscle cell (VSMC) growth that contributes to vasoconstriction. However, FR that promote VSMC growth are inhibited by GSH and reduced by LA. Topically applied LA may be even more efficient in this regard. Either LA or dihydrolipoic acid can rejuvenate other antioxidants (e.g., ascorbic acid and α-tocopherol) and raise intracellular GSH levels. As with most antioxidants, both can have a prooxidant effect if used in inappropriate levels; in this regard LA is safer, and also more effective at scavenging the hydroxyl radical than the reduced form dihydrolipoic acid. LA also increases intracellular GSH levels.

LA is of critical physiological importance within the cell, particularly within the mitochondrion. It is a proglutathione antioxidant. It is both water and lipid-soluble with a low molecular weight so that it readily passes through cell membranes and crosses the blood-brain barrier. Within cells and tissues, the salt form (α-lipoate) is reduced to dihydrolipoate, which is exported to the extracellular medium; hence, antioxidant protection is afforded to both intracellular and extracellular environments.

LA is an essential dehydrogenase co-factor in energy metabolism, a GSH sparing antioxidant and a substrate for GSH synthesis. It thereby increases cellular reduced GSH. This sparing effect depends in part by reducing cystine to cysteine. The cysteine thus formed is utilized for GSH synthesis. LA acts to normalize GSH levels rather than to increase it beyond physiological levels.

LA can prevent glutamate induced cellular damage (neuroprotection). This may prove to be clinically important in chronic glaucoma wherein glutamate induced neuronal/glial damage in the trabecular meshwork and ciliary body may disturb normal autoregulatory intraocular pressure control and indeed be a causative factor in the disastrous optic neuropathy of chronic glaucoma.

Transcription factor NF-kappaB is a cell-signaling pathway. While high concentrations of N-acetyl cysteine (NAC) can inhibit NF-kappaB activation, low concentrations of LA have a similar effect. These results suggest that some of the efficient antioxidant properties of LA may lie in its suppression of NF-kappaB activation.

Reduced GSH is a cofactor for the glyoxalase system, a metabolic pathway that catalyses the detoxification of α-oxoaldehydes (RCOCHO) to corresponding aldonic acids (RCH(OH)CO₂H). This protects cells from α-oxoaldehyde mediated formation of advanced glycation end products (AGE). AGE are implicated in a wide variety of ocular aging disorders including chronic glaucoma and cataract. Studies have found that incubation of cultured bovine aortic endothelial cells with AGE albumin results in decreases of GSH and ascorbic acid levels. This increased cellular oxidative stress leads to the activation of NF-kappaB and promotes the up regulation of various NF-kappaB-controlled genes, including endothelial tissue factor. However, the addition of LA before endothelial exposure to AGE albumin completely prevents depletion of GSH and ascorbic acid by inhibiting the release and translocation of NF-kappaB from the endothelial cytoplasm into the nucleus. Because LA reduces this AGE-induced, NF-kappaB mediated transcription, the expression of endothelial genes such as tissue factor and the vasoconstrictor ET-1 is reduced.

There is experimental evidence suggesting that the R stereoisomer of LA can prevent cataract development. The S isomer was not effective and the racemic mixture less than one-half as effective as the R isomer. The stereospecificity relates to selective uptake of R-lipoic acid by the lens. LA administration increases lenticular levels of the thiol contributor and of the GSH predecessor, cysteine. Cysteine is a rate-limiting precursor of GSH synthesis. It is generated from the extracellular thiol/disulfide exchange reaction of cystine and GSH (GSH+cystine<−−>cysteine+cysteine−GSH disulfide). This reaction creates an important ophthalmic protection because the ciliary body in particular contains a very active monooxygenase system prone to FR generation. These FR, combined with those produced via the cyclooxygenase pathway, result in ocular damage through oxidative stress.

As mentioned above, the retinal photoreceptor rhodopsin itself may be a photodynamic agent that initiates FR formation. Rhodopsin probably initiates FR formation via earlier-described mechanisms. FR produced via the cyclooxygenase pathway, on the other hand, may cause damage through oxidative stress-mediated vascular constriction. Both of these are logical contributors to the optic neuropathy of chronic glaucoma.

The protective antioxidative capacity of the youthful and healthy ciliary body is fortunately very high (via SOD and GSH: The peroxidation process in particular is countered by these defensive enzyme systems and antioxidants.)—Unfortunately much of this protection is lost with age and disease as available levels of required elements that underpin this defense systems fall. And a clear relationship appears to exist between unmodulated aqueous increases of H₂O₂-derived toxic FR (e.g., OH⁻) and the development of various age-related or disease-related ocular pathologies such as chronic glaucoma, cataract, corneal incompetencies of age and lacrimal gland dysfunction. Ensuring that appropriate levels of elements required for the restoration of more youthful or healthy defense mechanisms normally in place in the eye, is one of the rational bases for the invention.

C. cNO/ET-1 Balance

Components of the invention (including precursors, derivatives, homologs and analogs) with a primary effect on cNO/ET balance, one or more may be included in a product formulation of the invention, are:
1) L-arginine (including precursors and analogs)
2) Tetrahydrobiopterin (including precursors and analogs)
3) Folate (including precursors and analogs)

Preferred

| | |
|---|---|
| L-arginine | 0.017% to 5.487% |
| Tetrahydrobiopterin | 0.026% to 4.00% |
| Folate | 5.0E−08 to 0.158% |

A balanced biochemistry of NO and ET-1 mediates local ophthalmic blood flow and vascular autoregulation. Endothelium-derived NO released under basal conditions significantly regulates flow to the ophthalmic microcirculation, playing an important protective role against vasospasm and deleterious chronic vasoconstriction. In contrast, while ET-1 at low, physiological levels has some transient indirect vasodilator effects via the release of certain prostaglandins, it has more deleterious and potent, long lasting vasoconstrictor properties mediated through ETA receptors.

NO achieves its vasodilator effect by activating guanylate cyclase and increasing guanosine monophosphate (cGMP) within the VSMC and capillary pericytes. cGMP, in turn, produces relaxation and dilatation of these vessels.

Avoiding pathological vasoconstriction is of critical importance in preventing tissue hypoxia. Because a 10⁴ relationship exists between vascular diameter and flow (Poiseuille's law), decreasing the diameter of a vessel by 50% results in a dramatic 94% reduction of flow. The reoxygenation tissue damage, which follows ET-1 vasoconstriction, can be avoided if there is adequate L-arginine (the substrate for NO production) and tetrahydrobiopterin (the cofactor required for NO production—discussed later) in the tissue prior to anoxia. This finding underlines the importance of consistent, prophylactic use of the invention to prevent chronic glaucoma optic nerve damage, which is essentially irreversible once it has occurred.

ET-1 is formed within and secreted by endothelial cells in the anterior and posterior segments of the eye. It is released at pathological levels by aged or unhealthy dysfunctional endothelial cells or in the presence of local accumulations of endothelial leukocytes or platelets. Pathological levels of ET-1 and ET-1 receptor mRNA are present and are causal factors of the optic neuropathy in chronic glaucoma. Studies have shown that intravitreal ET-1 decreases optic nerve head blood flow.

ET-1 induces trabecular contraction and thereby increases resistance to aqueous outflow, thus increasing IOP. NO has the opposite effect, causing trabecular relaxation and lowering of the IOP. Elevated IOP enhances the production of NO in human trabecular cells further defining NO as a physiological mediator in the regulation of IOP. In chronic glaucoma, cNOS and NO levels are decreased in the trabecular meshwork and ET-1 is increased.

The NO/ET-1 balance is a physiologically balanced teeterboard. Elevated ET-1 decreases NO directly, and ET-1 induced hypoxia inhibits the expression of cNOS (the enzyme involved with NO production) via transcriptional and posttranscriptional mechanisms. Physiologically increased NO (via cNOS) decreases ET-1 by inhibiting ET-1 mRNA expression. The balance is clinically, not merely theoretically, important: Aging progressively tips the balance toward increased ET-1. During aging NO decreases, ET-1 increases and the potentiating effects of even very, low concentrations of ET-1 on VSMC contractions are augmented. The invention is designed to restore and maintain an appropriate NO/ET-1 balance.

L-arginine is the essential substrate for NOS generation of NO. It can be administered directly or via one of its precursors, L-ornithine, L-citrulline or proline+glutamine. L-arginine←→L-citrulline is reversible, with L-arginine being recycled via the argininosuccinate pathway.

Vasoconstrictive responses to ET-1 can be reversed with L-arginine. Long-term L-arginine supplementation improves small-vessel endothelial function because of this decrease in ET-1. NO derived from L-arginine inhibits ET-1 release, and thereby decreases ET-1 induced protein kinase C (PKC), leukocyte adhesion, platelet aggregation, superoxide generation, expression of vascular cell adhesion molecules, monocyte chemotactic peptides, and smooth muscle cell proliferation. L-arginine prevents, even reverses endothelial dysfunction associated with aging and ischemia/reperfusion injury and limits apoptosis. It lowers IOP mainly through the formation of NO.

BH4 is an essential cofactor for NOS and is also a scavenger of oxygen-derived free radicals. A deficiency of BH4 leads to reduced NO production and increased superoxide formation. Supplementation with L-arginine alone will inhibit this generation of superoxide by NOS. The effect of L-arginine on levels of superoxide, however, is less dramatic than that caused by tetrahydrobiopterin (BH4). And, in combination, L-arginine and BH4 cooperatively have the greatest inhibitory effect on superoxide generation. Used alone BH4 prevents vasoconstriction. BH4 metabolism occurs in ocular tissues, including the ciliary body and retina. The level of synthesis of BH4 is lower in the lens than in the above two ocular tissues, which may be a factor in cataract development.

Supplying this cofactor individually or as one component of a topical ophthalmic formulation is a novel approach to the favorable modulation of several pathologies of ciliary body, trabecular, retinal and optic nerve cellular dysfunction.

Folic acid and vitamin B12 appear to be required for the biosynthesis of BH4. The etiopathogenesis of folate deficiency in neurological disorders involves the regeneration of BH4, which is an essential cofactor in the brains of mammals. One should note here that developmentally, anatomically, and functionally the eye is merely an extension of the central nervous system. Therefore, it should not come as a surprise that there be a similar relationship between various ocular neuropathies and the nexus of folic acid/vitamin B12 with BH4.

It is clear that ET-1 exerts a wide range of biological actions besides its dominant vasoconstrictor effect. As one more example: It also increases mRNA levels of collagen I and of fibronectin, each of which may contribute to ocular pathologies such as elevated IOP.

For the above multitude of reasons, the invention focuses heavily upon maintaining or reestablishing appropriate levels of those elements known to be necessary as precursors, active biofactors or cofactors in positively modulating the NO/ET-1 balance. And it delivers them in an efficient and unique fashion.

D. Calcium Wave Signaling (& Associated Neuronal Excito-Toxicity)

Calcium (Ca⁺²) is the most highly regulated ion in nature. Normal physiology restricts it to a 10,000 times lower concentration intracellularly compared to its extracellular concentration. Membrane pumps and ion channels, and intracellular stores all control the entry and exit of this ubiquitous second messenger ion. The amount and rate of change of free extracellular Ca⁺² is also moderated by many intracellular calcium-binding sites that buffer its levels. Ca⁺² metabolism is complex and singularly important in health and disease.

This essential intracellular and intercellular messenger delivers its messages via frequency modulated waves and oscillations, entering the cytoplasm from both intracellular and extracellular sources.

While the exact function of this complex Ca⁺² activity is not completely understood it is believed that the waves and oscillations are frequency dependent codes. This increases the signal-to-noise ratio allowing Ca⁺² to be used as an efficient intracellular and intercellular messenger, while avoiding the toxic effect of prolonged elevated Ca⁺². Intracellular waves coordinate the response of an entire cell to a local stimulus while intercellular waves serve to coordinate the response of a group of cells.

As one practical example of information encoding, note that the encoding of information by frequency rather than by amplitude accounts for the much higher information content of audio FM compared to audio AM. For cellular physiological processes, resolution is more important than amplitude. Increasing the amplitude of information without maintaining the clarity of resolution leads to pathology. In the case of Ca⁺² it directly leads to excess ET-1, to mitochondrial dysfunction and to cell death. Formulations of the invention assemble complementary components to effectively modulate Ca⁺² signaling and avoid the large amplitude Ca⁺² waves that are associated with the clinical ocular pathologies targeted by the invention.

All of this complex modulated signaling requires a fine balance between Ca⁺² flow patterns of the cell membrane, the plasma and the endoplasmic reticular substances and Ca⁺² flow patterns within and from the cytoplasm and nucleus. Enzymes control many of these orchestrated Ca⁺² waves and magnesium (Mg⁺²) is a key cofactor of these activities. Similarly, 17 α-estradiol and the amino acid taurine improve cellular Ca⁺² dynamics and are functionally complementary to Mg⁺². For these reasons Mg⁺², taurine and 17 α-estradiol are included in the invention.

When Ca⁺² signaling becomes dysfunctional it can cause vasoconstriction, vascular smooth muscle proliferation and apoptosis, as is seen in chronic glaucoma. Homocysteine amplifies Ca⁺² waves by inducing excessive Ca⁺² release from the endoplasmic reticulum; this results in vasoconstriction, vascular smooth muscle proliferation, and increased deposition of extracellular matrix. Folate effectively metabolizes homocysteine, ameliorating this effect.

Components of the invention (including precursors, derivatives, homologs and analogs) with a primary effect in the area of Calcium Wave Modulation (& Associated Neuronal Excitotoxicity). One or more may be included in a product formulation of the invention:
1. Magnesium
2. Taurine
3. 17 α-estradiol

Preferred

| | |
|---|---|
| Magnesium | 0.017% to 4.725% |
| Taurine | 0.004% to 3.886% |
| 17 α-estradiol | 0.008% to 2.962% |

Mg⁺², by a variety of mechanisms, functions both intracellularly and extracellularly to optimize the cytoplasmic free Ca⁺² level. Excess cytoplasmic free Ca⁺² leads to overproduction of ET-1 with resultant vasoconstriction, platelet aggregation and apoptosis. For these reasons correction of Mg⁺² deficiency exerts antihypertensive, antithrombotic and anti-atherosclerotic effects. Mg⁺² accomplishes this without interfering with normal Ca⁺² intracellular signaling. In comparison, pharmaceutical calcium channel blockers have gross, amplitude driven, effects on Ca⁺² cellular signaling, the undesirable effects of which are described in more detail above. Mg⁺² deficiencies are widespread in the elderly glaucoma population.

Taurine is a conditionally essential amino acid, which is not utilized in protein synthesis, but rather is found free or in simple peptides. Taurine deficiency is associated with a variety of pathologies, including retinal degeneration. Taurine is important for the modulation of cellular Ca⁺² levels, cell membrane stabilization and osmoregulation. Clinically, taurine has been used with varying degrees of success in the treatment of several conditions, including macular degeneration and Alzheimer's disease. There is evidence that taurine has an effect on reducing toxic effects on neurons.

Taurine modulates Ca⁺² signaling, increasing cytosolic Ca⁺² transients in cardiac cells (positive ionotropic activity), and reducing cytosolic Ca⁺² in other cells. Similar to Mg⁺², taurine lowers elevated blood pressure, retards cholesterol-induced atherogenesis, prevents arrhythmias, and stabilizes platelets and cell membranes. Its favorable modulation of Ca⁺² signaling complements Mg⁺². It also competitively blocks the glutamate NMDA receptor and/or glutamate release at synapse lessening glutamate neuronal excitotoxicity.

Elevated Homocysteine (Hcy), which is common in the general population, is established as a common, significant risk factor for neuronal excitotoxicity and vasomotor dysregulation. It induces pathological levels of Ca⁺² release from intracellular stores resulting in overproduction of ET-1, vasoconstriction, vascular smooth muscle proliferation and increased deposition of extracellular matrix. Hcy induced Ca⁺² waves can be inhibited by calcium channel blockers or by folic acid. Chronic glaucoma is often associated with vasomotor dysregulation that can be caused or aggravated by Hcy.

Hcy induces excitotoxicity by acting as an agonist at the glutamate-binding site of the N-methyl-D-aspartate receptor, resulting in a disproportionate flow of Ca⁺² into neurons. Accordingly, this Hcy-induced neurotoxicity through over stimulation of N-methyl-D-aspartate receptors probably contributes to pathogenesis of the ciliary body, retina, optic nerve, and trabeculum in the presence of even modest hyperhomocysteinemia.

In addition to its beneficial affect on the mitochondrial membrane (see above) the estrogen stereoisomer, 17 α-estradiol, has other valuable clinical features of importance to the invention. It modulates calcium channels, can attenuate glutamate excitotoxicity and (perhaps) as a free radical scavenger prevents acinar cell death in the lacrimal gland. These functions are independent of the β-estradiol (estrogen) receptor. 17 α-estradiol, which has no female hormone activity, provides an antiapoptotic effect. The antiapoptotic effect is independent of female hormone activity.

### II. Clinical Targets

This section describes the pertinent biochemistry of each of the primary clinical intraocular ophthalmic targets of the invention and the influence upon each by the invention. These targets are as follows:
A. Chronic Glaucoma
B. Cataract
C. Ocular and Periocular Aging

There are some etiologic similarities for the conditions targeted by the invention, which may include dysfunctional bioenergetics, cNO/ET-1 imbalance, excessive free radicals and ineffectively modulated intracellular and intercellular calcium signaling waves. The complexity of these metabolic subsystems and their interdependence demands multiple administered components to prevent or ameliorate the targeted conditions. A single therapeutic “silver bullet” is unrealistic when dealing with the intricacy and nonlinearity of these conditions. The invention addresses the complex pathophysiology of the involved metabolic subsystems with multicomponent scientifically orchestrated formulations.

A. Chronic Glaucoma

Chronic glaucoma is a condition whose genesis and progression are dependent upon IOP levels, and risk factors independent of the IOP. Many patients develop progressive glaucoma damage without any elevation of IOP: Seventy percent of these ‘low tension’ or ‘normotensive’ glaucoma patients have various cardiovascular or cerebrovascular risk factors. Vasospasm is common in this group. Myopic patients are more vulnerable to glaucoma damage, and for these patients vascular factors play an important role.

In glaucoma the definitive blinding pathology is in the posterior segment of the eye—the optic nerve. There is often associated pathology in the anterior segment of the eye—the trabecular meshwork. In chronic glaucoma, dysfunctional tissue in both the anterior and posterior segment results in part from metabolic pollution (viz. free radicals, excessive ET-1, excitotoxins, homocysteine) dispersed from the ciliary body and retinal vasculature into the aqueous and vitreous.

Current non-surgical treatments of COAG are based upon a limited number of biochemical approaches and focus exclusively upon reducing intraocular pressure (IOP). These include:
Enzyme poisons that reduce aqueous production by blocking carbonic anhydrase in the ciliary body;
Parasympathomimetics that induce gross muscular contraction of the iris and scleral spur;
Beta blocking agents which indirectly reduce the production of aqueous humor by inhibiting beta adrenergic innervation of the ciliary body;
Newly introduced topical prostaglandin analogs, which increase the outflow of aqueous through the trabeculum by widening the intra-trabecular space and, perhaps, by reducing platelet aggregation. However, there are some indications that their use is associated with episodic anterior uveitis, and they may be contraindicated in patients with a history of uveitis or prior ocular surgery.

While individually effective in reducing IOP, none of these treatment modes consistently stops the progression of glaucoma and each has significant and unavoidable, potential or demonstrable, local or systemic side effects or toxicities that directly contraindicate their use, reduce patient compliance or are worrisomely interactive with other systemic drugs. Because the invention uses physiological molecules in precise formulations, these toxicities and interactivities are practically non-existent with its clinical use, and as a result, contraindications are also almost non-existent. However, this lack of adverse effects in no way indicates a weakness of effectiveness.

Mitochondrial Bioenergetics

Dysfunctional mitochondrial bioenergetics, as discussed above, results in pathological apoptotic cell death. In chronic glaucoma this is evident in optic nerve neurodegeneration and in trabecular meshwork pathology. Separately and normally, there is also gradual loss of cells in the human trabecular meshwork during aging. This process is accelerated in patients with primary open-angle glaucoma. Studies in a glaucoma rat model have shown that mitochondrial membrane potential of cells in the retinal ganglion cell layer is reduced by 17.5% in regions surrounding areas of focal cell loss compared with control, normal eyes and showed cell nuclei at various stages of apoptosis, from initial DNA condensation to fragmentation after 3.5 months of elevated IOP.

Free Radical Moderation (and Glutathione Maintenance)

FR produced by the ciliary body circulate in the aqueous humor and damage the cells of the trabecular meshwork as the aqueous percolates through this tissue while exiting the eye. The content of lipid peroxides in aqueous increases in parallel with the severity of chronic glaucoma. Glaucomatous optic neuropathy is at least in part the result FR release from the retinal and local optic disc vasculature. Glutathione, a principal intracellular defense against reactive oxygen species generated during mitochondrial respiration, is decreased in glaucoma. FR cause further antioxidant vascular endothelium abnormalities neutralize NO-mediated vasodilatation and increase reactivity to vasoconstrictors, a feed forward pathological cycle. In the long-term, elevated FR cause cumulative damage to neurons and to Schwann cells, in addition to their deleterious short-term effects on nerve blood flow. This results in endoneural hypoxia, which is responsible for early nerve conduction deficits. This is true for the trabecular meshwork as well as the retina. Antioxidant treatment corrects the blood flow deficit and promotes normal endoneural oxygenation.

cNO/ET-1 Balance

The physiology of NO is varied and complex. The complicated, often interrelated, physiological and pathological functions of NO and the extensive distribution of the NOS system creates opportunities for the use of NO-related agents, not only in the control of IOP in primary open-angle glaucoma (POAG) but also in the lessening of pathological vasoconstriction in normotensive glaucoma (NTG); and possibly in the prevention or improvement of glaucomatous optic neuropathy.

The cNO/ET-1 balance is adversely disturbed in chronic glaucoma; there is excess ET-1 and reduced NO. ET-1 is increased above normal in both the aqueous and vitreous. In the aqueous, excess ET-1 causes an abnormal relationship between the contraction of the ciliary muscle (CM) at its insertion into the scleral spur, and contraction within the trabecular meshwork (TM). Under normal conditions, CM contraction increases aqueous outflow through the trabecular meshwork and decreases IOP, while contraction of trabecular cells decreases meshwork outflow and elevates IOP. Normally in both the CM and the TM there is a constant release of NO—the TM system is the more sensitive. This NO relaxes the contractile trabecular elements of the eye, increases aqueous outflow and reduces IOP. Unfortunately levels of NO in the trabecular region of eyes of glaucoma patients are lower than in the eyes of non-glaucoma patients. Aging and atherosclerotic dysfunction of the vascular endothelium of the trabecular meshwork further reduce local levels of NO because of reduced levels of cNOS or the NO substrate, L-arginine. With unbalanced levels of ET-1 the net effect is trabecular contraction, impedance of aqueous outflow through the TM and elevation of the IOP.

In the posterior segment of the eye, at the optic nerve, excess ET-1 causes vascular dysregulation and possible ischemia that can lead to the retinal ganglion cell death characteristic of glaucoma. In contrast to ET-1, cNO through activation of the guanylate cyclase pathway normally maintains the optic nerve vasculature (including the capillaries) in relaxed, mid-dilation. In the laboratory, induced elevations of aqueous ET-1 levels produce optic nerve collapse. This is a vicious cycle that functionally underlies the slow optic nerve collapse that leads to the final blindness that characterizes glaucoma.

Dysregulation of calcium signaling waves (see above) is involved with glaucoma optic nerve damage from vasoconstriction/ischemia and retinal ganglion cell excitotoxicity. In the former (vasoconstriction) hypoxia can lead directly to cell death or indirectly to cell necrosis via reperfusion injury. In the optic nerve three neural areas are particularly vulnerable to hypoxia:
The microglial ganglion cells.
The transiting axonic neurons.
The neural innervation of the trabecular meshwork.

A reduction in optic nerve oxygen delivery may follow acute or chronic, segmental or widespread, vascular spasm or prolonged constriction secondary to a physical reduction in the vascular lumen. This luminal reduction (vasoconstriction) may be caused by or associated with hypertrophy of the vascular muscle wall (the media), the accumulation of atherosclerotic plaque, platelet agglutination and/or local inflammatory swelling and leukocytic accumulation. Any and all of these findings may, and usually do, occur with aging. Although vascular insufficiency at specific tissue sites is widely variable and not predictable with certainty, the fact that most chronic glaucoma patients are over 50 years old makes the frequency of concurrency of these risk factors and the correlative frequency of vascular insufficiency high in chronic glaucoma.

Exposure of patients to calcium channel blockers has resulted in an improvement of some glaucomatous visual fields. Vascular endothelial production of ET-1 is dependent upon cytosolic Ca²⁺ influx via transmembrane Ca²⁺ channels. Calcium channel blockade reduces this Ca²⁺ influx and reduces the production of ET-1. Not surprisingly, a reduction of IOP has been observed as a side effect in glaucoma patients using calcium channel blockers for systemic hypertension. However, prescribing at therapeutic levels, systemic doses of these drugs to non-hypertensive glaucoma patients subjects the optic nerve to a risk of hypoxia secondary to iatrogenic hypotension, and additionally severely disrupts inherent transmembrane Ca²⁺ modulation. Chronic exposure to hypoxia and FR additionally lead to accelerated apoptosis of the delicate neural network of the trabecular meshwork.

In excitotoxicity, excessive Ca²⁺ waves occur through glutamate stimulated postsynaptic NMDA receptors resulting in a caspase cascade terminating in retinal ganglion cell apoptosis (discussed above). Glutamate (an excitotoxic amino acid known to induce neuronal apoptosis) is elevated in the vitreous in eyes with chronic glaucoma. In addition to its role in stabilizing the mitochondrial membrane, α-estradiol attenuates glutamate excitotoxicity by a mechanism that is independent of the ability of the steroid to bind the estrogen receptor.

Until now little attention and less awareness has been apparent for the existence of chronic neuropathology of the innervation of the trabecular meshwork in chronic glaucoma patients. The invention also addresses this glaucoma parameter.

The treatment of glaucoma by restoring normal or youthful defenses to these areas of deficiency by the topical delivery of critical components known to be lacking or inadequate is unique to this patent.

The ophthalmic solution percentages illustrated in the following example may be adjusted up or down, component-by-component, or overall to achieve maximum therapeutic effectiveness according to the ophthalmic delivery method chosen.

### EXAMPLE 1

1.25% Ophthalmic Topical Dosage Form: Glaucoma

| | | |
|---|---|---|
| Coenzyme Q10 | 0.23% | Mitochondrial Bioenergetics |
| | | (& Excitotoxicity Defense) |
| R-alpha-lipoic acid | 0.22% | FR Moderation (& Glutathione |
| | | Maintenance) |
| Tetrahydrobiopterin | 0.30% | cNO/ET-1 Balance |
| 17 alpha-estradiol | 0.20% | Mitochondrial Bioenergetics & |
| | | Ca²⁺ Signal Modulation |
| Glutathione | 0.30% | FR Moderation (& Glutathione |
| | | Maintenance) |

These components interrelate in a complementary or synergistic manner to reduce dysfunctional apoptosis and neurotoxicity in the region of the trabecular meshwork, and in the retinal ganglion cells of the optic nerve. An example of synergy is that in the presence of glutathione (GSH) the neuroprotective potency of estradiol is increased 400-fold. The invention reduces several glaucoma risk factors and prevents or ameliorates the visual loss of chronic glaucoma. Other components from a list contained within the patent may be substituted for the above, or may be added, depending on the formulation most appropriate for the delivery system utilized, the tissue levels attainable, the nature of the disease, the stage of the disease or considerations of patient tolerance.

B. Cataract

The crystalline lens of the eye is continuously bathed by the aqueous humor produced by the ciliary body. Aqueous humor contains pollutants, especially FR and ET-1, which increase with age and whose production is accelerated by coexisting anterior segment pathological processes like chronic glaucoma and inflammation. The lens is particularly vulnerable to FR-induced apoptosis. Gradually over decades, sometimes less, the crystalline lens succumbs to FR, progressively loses its transparency and the patient's vision declines.

Mitochondrial Bioenergetics

Mitochondrial bioenergetic dysfunction with ensuing apoptosis is associated with senile cataract development.

There are two main apoptotic signaling pathways: a death receptor-dependent (extrinsic) pathway and a mitochondrion-dependent (intrinsic) pathway. The mitochondrion harbors both antiapoptotic (Bcl-2, Bcl-XL) and apoptotic factors (Bax, Apaf-1, cytochrome c). Its permeability transition pore (mPTP) is the main trigger of cell suicide. A respiratory chain constituent that is a component of the invention, CoQ10, counteracts mPTP opening.

Additionally, the mitochondrion, the most important intracellular source of FR, when dysfunctional, produces excess FR that damages its own DNA in a feed-forward mechanism; and causing diseases associated with aging like senile cataracts.

Free Radical Moderation (& Glutathione Maintenance)

The crystalline lens is submersed in a continuous flow of aqueous secreted by the ciliary body. During aging, the ciliary body has reduced mitochondrial respiration efficiency, which produces increasing amounts of the superoxide radical, which in turn sets off a self-propagating chain of FR production. Glutathione (GSH), the main antioxidant of the lens is thereby progressively decreased, creating a yet another feed-forward cycle leading to ever-increasing FR being introduced by the ciliary body into the aqueous humor. In time this FR exposure of lenticular cells leads to apoptosis and individual cellular opacification. Ultimately the accumulation of opaque apoptotic cells causes sufficient loss of overall lens transparency to interfere with vision and be termed a cataract. In contrast to most tissues, lenticular apoptotic cells are not efficiently disposed of by macrophages, thus the opaque corpses remain within the crystalline lens to permanently cloud vision.

There are three aspects of the functional role of GSH in cataract formation: 1) GSH maintains protein thiols in the reduced state, thus preventing the formation of high molecular weight protein aggregates, which are the basis for light scattering and lens opacification; 2) GSH protects cell membranes from peroxidation and altered cation transport and permeability; 3) GSH reduces FR mitochondrial effects and apoptosis.

Tetrahydrobiopterin (BH4) content is decreased in human senile cataracts as compared with age-matched clear human lenses. The loss of BH4 results in lenticular proteins more susceptible to oxidation and contributes to formation of the light scattering, high molecular weight protein aggregates characteristic in cataract development.

Since the lens is avascular, virtually all pathological influences are derived from the aqueous humor. To prevent or slow cataract development FR secretion from the ciliary body must be reduced, GSH levels within the lens must be maintained and mitochondrial integrity must be preserved. The invention is designed to meet these goals.

The ophthalmic Topical Dosage Form Percentages illustrated in the following example may be adjusted up or down, individually or as a group, to achieve maximum therapeutic effectiveness according to the ophthalmic delivery method chosen.

### EXAMPLE 2

1.25% Ophthalmic Topical Dosage Form: Cataract

| | | |
|---|---|---|
| Coenzyme Q10 | 0.23% | Mitochondrial Bioenergetics |
| | | (& Excitotoxicity Defense) |
| R-alpha-lipoic acid | 0.22% | FR Moderation (& Glutathione |
| | | Maintenance) |
| Tetrahydrobiopterin | 0.30% | cNO/ET-1 Balance |
| 17 alpha-estradiol | 0.20% | Mitochondrial Bioenergetics & |
| | | Ca²⁺ Signal Modulation |
| Glutathione | 0.30% | FR Moderation (& Glutathione |
| | | Maintenance) |

These components interrelate in a complementary or synergistic manner to reduce dysfunctional apoptosis in the crystalline lens of the eye, thereby reducing cataract risk factors and preventing or ameliorating visual loss. Other components from a list contained within the patent may be substituted for the above or may be added depending on the formulation most appropriate for the delivery system utilized, the tissue levels attained, the nature of the disease, the stage of the disease or patient tolerance considerations.

C. Ocular and Periocular Aging

The aging process is without doubt the most complex physiological collection of events that gather around any biological system, and consequently it represents the most challenging therapeutic target. No one can claim to end this congeries of events: The most one can achieve is an attenuation of the speed at which the events occur and some modification of (reduction or defense against) cumulative risk factors that influence that rate of decline. However, because of the vast number of variables involved, any therapeutic approach must he tightly focused upon specific nodes of biological instability that may be positively altered, improved or stabilized.

Identifiable ophthalmic diseases are more frequently diagnosed in the aged. These include cataract, chronic glaucoma, macular degeneration, and dry eye syndrome, among others. These specific ocular and periocular problems are underpinned however by global disequilibria in fundamental cell membrane and mitochondrial function. The invention addresses both levels of the ocular and periocular aging process by directly providing to the eye support for global cellular physiological failures and support for more organ specific failures (e.g., cataract, trabecular meshwork dysfunction, reduced corneal respiratory physiology, increases in intraocular pressure and limits on ocular pressure autoregulation, cumulative vascular vasoconstrictive changes in the anterior and posterior chambers, lacrimal gland acinar cell apoptosis, etc).

The commonality of cause of these age-related processes is significant in the areas of disturbed mitochondrial bioenergetics, FR excess, cNO/ET-1 imbalance and unbridled Ca²⁺ wave signaling. These are discussed above.

The ophthalmic Topical Dosage Form Percentages in the following example may be adjusted up or down, individually or as a group, to achieve maximum therapeutic effectiveness according to the ophthalmic delivery method chosen.

### EXAMPLE 3

1.25% Ophthalmic Topical Dosage Form:
Ocular and Periocular Aging

| | | |
|---|---|---|
| Coenzyme Q10 | 0.23% | Mitochondrial Bioenergetics |
| R-alpha-lipoic acid | 0.22% | FR Moderation (& Glutathione |
| | | Maintenance) |
| Tetrahydrobiopterin | 0.30% | cNO/ET-1 Balance |
| 17 alpha-estradiol | 0.20% | Mitochondrial Bioenergetics & |
| | | Ca²⁺ Signal Modulation |
| Glutathione | 0.30% | FR Moderation (& Glutathione |
| | | Maintenance) |

Other components from a list contained within the patent may be substituted for the above, or may be added, depending on the formulation most appropriate for the delivery system utilized, the tissue levels attainable, the nature of the disease, the stage of the disease or considerations of patient tolerance.

### SUMMARY

The cell is a complex physicochemical system with nonlinear metabolic functions, each of which has a sensitive dependence on initial conditions. A system of networked cells exhibits extraordinarily complex behaviors in time and space. Such a biological system consists of an identifiable set of metabolic subsystems and nodes. Finally, there exists a web of interactions between these nodes and between the subsystems that constitutes a metabolic reaction network.

The invention defines the metabolic subsystems, which when disturbed are involved in causing various ocular pathologies that share these subsystems. The shared subsystems are: 1) mitochondrial bioenergetics, 2) free radical maintenance, 3) constitutive nitric oxide/endothelin-1 balance, and 4) calcium signaling (wave and oscillation).

The ocular conditions that occur when some or all of these shared subsystems are disturbed are: 1) chronic glaucoma, 2) cataract and 3) ocular (and periocular) aging. Since metabolic subsystems are shared, some of these clinical conditions may coexist, if one or several of the metabolic subsystems are dysfunctional.

Therapy directed toward one subsystem is unlikely to be as successful as that which favorably modifies all of the effected subsystems. The invention is based on this principal. Each pathophysiological condition has a multicomponent formulation designed for it; that particularly addresses the disturbed metabolic subsystems and their interrelationships. This affords the highest probability of therapeutic success while maintaining safety.

Stated somewhat differently, there are commonalities, which may be shared to various degrees by the clinical conditions targeted by the invention. These encompass dysfunctional bioenergetics, cNO/ET-1 imbalance, excessive free radicals and ineffectively modulated intracellular and intercellular calcium signaling waves. The complexity of these areas of metabolic subsystem dysfunctional causation and their interdependence, demands administration of multiple, orchestrated components to prevent or ameliorate the targeted conditions. A single therapeutic “silver bullet” is unrealistic when dealing with the complexity of these conditions. The invention addresses the etiologic commonality with multicomponent scientifically orchestrated formulations.

### DEFINITIONS

All terms appearing in this specification and the appended claims are used in the same manner as commonly recognized among those skilled in the technology and terminology of pharmacology. These terms are therefore used in accordance with their conventional definitions, except as otherwise noted. Further clarifications of some of these terms as they apply specifically to the invention are offered below.

“Unit dosage form” refers to a composition intended for a single administration to treat a subject suffering from a disease or medical condition. Each unit dosage form typically comprises each of the active ingredients of the invention plus pharmaceutically acceptable excipients. Examples of unit dosage forms in the invention are liquid solutions, ointments, creams, eyedrops, emulsions, suspensions or transmembrane or osmotic delivery systems. Treatment of the disease or condition may require periodic administration of unit dosage forms, for example: one unit dosage form two or more times a day, one every four hours or other interval, one per day or one every other day or other extended interval.

An “active agent” or “active ingredient” is a component of a dosage form that performs a biological function when administered or induces or affects (enhances or inhibits) a physiological process in some manner. “Activity” is the ability to perform the function, or to induce or affect the process. Active agents and ingredients are distinguishable from excipients such as carriers, vehicles, diluents, lubricants, binders, buffers and other formulating aids, and encapsulating or otherwise protective components.

“Delivery vehicle” is a composition, which comprises one or more active agents, and is designed to release the active agent in a particular fashion, either by immediately dispersing the agents, or by releasing the agents in a slow sustained fashion. The term encompasses ophthalmic gels, porous microspheres, microcapsules, cross-linked porous beads, polymers and particles, and liposomes that contain one or more active ingredients sequestered within internal cavities or porous spaces. The term also includes osmotic delivery systems, that include nonporous microspheres, microcapsules, and liposomes, and active agents dispersed within polymeric matrices and transmembrane delivery systems. In addition to the above Delivery Vehicles, the terms “Ocular Delivery and Ophthalmic Delivery” contemplate techniques and methods of delivery that include transmembrane systems, electrophoresis, retrobulbar injection, conjunctival and subconjunctival injection, solutions, gels, suspensions or ointments. A dosage form can include one or more delivery vehicles.

“Controlled” or “sustained” or “time release” delivery are equivalent terms that describe the type of active agent delivery that occurs when the active agent is released from a delivery vehicle at an ascertainable and manipulatable rate over a period of time, which is generally on the order of minutes, hours or days, typically ranging from about thirty minutes to about 3 days, rather than being dispersed immediately upon entry into the eye or upon contact with the tears. A controlled release rate can vary as a function of a multiplicity of factors. Factors influencing the rate of delivery in controlled release include the particle size, composition, porosity, charge structure, and degree of hydration of the delivery vehicle and the active ingredient(s), the pH of the environment (either internal or external to the delivery vehicle), and the solubility of the active agent in the physiological environment.

“Targeted” or “site-specific” delivery means that the pharmaceutical preparation is formulated to limit the release of its contents in an amount appropriate to the site where release occurs. The term refers in particular to the active agent, whose site-specific delivery implements the performance of the therapeutic function at a specific site within the body of the subject to whom the preparation is administered.

The phrase “therapeutically effective amount” means an amount sufficient to produce a therapeutic result. Generally the therapeutic result is an objective or subjective improvement of a disease or condition, achieved by inducing or enhancing a physiological process, blocking or inhibiting a physiological process, or in general terms performing a biological function that helps in or contributes to the elimination or abatement of the disease or condition.

### Composition of Ophthalmic Dosage Forms

1. Ophthalmic Solutions

The topical ophthalmic dosage form of the invention will optionally include one or more suitable and pharmaceutically acceptable inactive excipients, including but not limited to: preservatives from a group including benzalkonium chloride, methylparaben, edetate disodium, thimerosol, chlorbutanol; buffers from a group including sodium citrate, potassium chloride, magnesium chloride, sodium acetate, citric acid, sodium lactate; vehicles from a group including polyvinyl alcohol, hydroxy methylcellulose, cetyl alcohol, carboxymethylcellulose, hydroxy-propylenemethyl cellulose; pH adjusters from a group including sulfuric acid, hydrochloric acid, sodium hydroxide, monosodium or disodium phosphate; purified water USP; poloxamer 407 or 188, polysorbate 80; polyoxyethylene polyoxypropylene compound; mineral oil USP and similar products.

This dosage form may also include proprietary ophthalmic suspensions and emulsions, whether water or lipid soluble, and be capable of immediate or sustained delivery. Examples of these classes of proprietary ophthalmic delivery methods and compositions are disclosed in Bowman et al, U.S. Pat. No. 5,977,171 (“Sustained Release Emulsions”; Bowman et al, U.S. Pat. No. 6,159,458 (“Sustained Release Ophthalmic Compositions Containing Water Soluble Medicaments”); Patel et al, U.S. Pat. No. 5,474,764 (“Alkaline Ophthalmic Suspensions”); Chandrasekaran et al, U.S. Pat. No. 5,188,826 (“Topical Ophthalmic Suspensions”); Davis et al, U.S. Pat. No. 5,192,535 (“Ophthalmic Suspensions”); Bowman et al, U.S. Pat. No. 5,767,153 (“Sustained Release Emulsions”) and Bowman et al, U.S. Pat. No. 6,265,444 (“Ophthalmic Composition”), incorporated herein by reference in their entirety.

The above inactive excipients serve a variety of functions as carriers, vehicles, diluents, binders, preservatives, buffers, pH adjusters, emulsifiers and other formulating aids as briefly listed above and are currently in wide use in ophthalmic pharmaceutical products manufactured under GMP standards.

The topical ophthalmic dosage forms of The invention can be formulated for administration at a rate of one unit dosage form daily or two or more unit dosage forms four times daily, or one or more unit dosage forms at intervals longer that one day. The amounts of the primary components of the topical ophthalmic dosage form of the invention will vary, although in preferred preparations the components are present in amounts lying within certain ranges. These are shown above in relation to 1) metabolic subsystems and 2) specific targeted pathophysiological targets.

2. Ophthalmic Ointment

An ophthalmic ointment dosage form for more prolonged delivery of the formulations or for use at bedtime will optionally include one or more suitable and pharmaceutically acceptable inactive excipients, including but not limited to: chlorbutanol, polyethylene mineral oil gel, white petrolatum USP, mineral oil USP, petrolatum and lanolin alcohol, purified water USP, polyvinyl alcohol gel and similar products.

This dosage form may also include proprietary ophthalmic suspensions and emulsions, whether water or lipid soluble, and be capable of immediate or sustained delivery. Examples of these classes of proprietary ophthalmic delivery methods and compositions are disclosed in Bowman et al, U.S. Pat. No. 5,977,171 (“Sustained Release Emulsions”; Bowman et al, U.S. Pat. No. 6,159,458 (“Sustained Release Ophthalmic Compositions Containing Water Soluble Medicaments”); Patel et al, U.S. Pat. No. 5,474,764 (“Alkaline Ophthalmic Suspensions”); Chandrasekaran et al, U.S. Pat. No. 5,188,826 (“Topical Ophthalmic Suspensions”); Davis et al, U.S. Pat. No. 5,192,535 (“Ophthalmic Suspensions”); Bowman et al, U.S. Pat. No. 5,767,153 (“Sustained Release Emulsions”) and Bowman et al, U.S. Pat. No. 6,265,444 (“Ophthalmic Composition”), incorporated herein by reference in their entirety.

The above excipients serve a variety of functions as carriers, vehicles, diluents, binders, preservatives, buffers, pH adjusters, emulsifiers and other formulating aids and are currently in wide use in pharmaceutical products manufactured under GMP standards.

The ointment dosage forms of the invention can be formulated for administration at rates of one unit dosage form daily or two or more unit dosage forms four times daily, or one or more unit dosage forms at intervals longer that one day. The amounts of the primary components of the ophthalmic ointment dosage form of the invention can vary, although in preferred preparations the components are present in amounts lying within certain ranges.

### REFERENCE LIST

The following references are incorporated herein by reference for all purposes legally capable of being served thereby.
(1) Austin R C, Sood S K, Dorward A M, Singh G, Shaughnessy S G, Pamidi S et al. Homocysteine-dependent alterations in mitochondrial gene expression, function and structure. Homocysteine and H2O2 act synergistically to enhance mitochondrial damage. J Biol Chem 1998; 273(46):30808-30817.
(2) El Idrissi A, Trenkner E. Growth factors and taurine protect against excitotoxicity by stabilizing calcium homeostasis and energy metabolism. J Neurosci 1999; 19(21):9459-9468.
(3) Giblin F J. Glutathione: a vital lens antioxidant. J Ocul Pharmacol Ther 2000; 16(2):121-135.
(4) Haefliger I O, Flammer J, Beny J L, Luscher T F. Endothelium-dependent vasoactive modulation in the ophthalmic circulation. Prog Retin Eye Res 2001; 20(2):209-225.
(5) Han D, Handelman G, Marcocci L, Sen C K, Roy S, Kobuchi H et al. Lipoic acid increases de novo synthesis of cellular glutathione by improving cystine utilization. Biofactors 1997; 6(3):321-38.
(6) Lass A, Sohal R S. Effect of coenzyme Q(10) and alpha-tocopherol content of mitochondria on the production of superoxide anion radicals. FASEB J 2000; 14(1):87-94.
(7) Lenaz G, Bovina C, Formiggini G, Castelli G P. Mitochondria, oxidative stress, and antioxidant defences. Acta Biochim Pol 1999; 46(1):1-21.
(8) Loeffler M, Kroemer G. The mitochondrion in cell death control: certainties and incognita. Exp Cell Res 2000; 256(1):19-26.
(9) Matthews R T, Yang L, Browne S, Baik M, Beal M F. Coenzyme Q10 administration increases brain mitochondrial concentrations and exerts neuroprotective effects. Proc Natl Acad Sci USA 1998; 95(15):8892-8897.
(10) Packer L, Tritschler H J, Wessel K. Neuroprotection by the metabolic antioxidant alpha-lipoic acid. Free Radic Biol Med 1997; 22(1-2):359-378.
(11) Vasquez-Vivar J, Hogg N, Martasek P, Karoui H, Pritchard K A, Kalyanaraman B. Tetrahydrobiopterin-dependent inhibition of superoxide generation from neuronal nitric oxide synthase. J Biol Chem 1999; 274(38):26736-26742.
(12) Wiederholt M, Sturm A, Lepple-Wienhues A. Relaxation of trabecular meshwork and ciliary muscle by release of nitric oxide. Invest Ophthalmol Vis Sci 1994; 35(5):2515-2520.

We claim:

1. A method for treating a patient for the prevention, management and clinical amelioration of chronic glaucoma, ocular aging, periocular aging and conditions giving rise thereto, said method comprising administering to said patient a unit dosage form comprising as an active ingredient a therapeutically effective amount of tetrahydrobiopterin.

2. The method of claim 1 wherein said dosage form is formulated as an eyedrop.

3. The method of claim 2 wherein said tetrahydrobiopterin is in an amount ranging from about 0.026% to about 4.00% by weight of said dosage form.

4. The method of claim 3 wherein said dosage form further comprises R-alpha-lipoic acid in an amount ranging from about 0.002% to about 3.15% by weight of said dosage form.

5. The method of claim 3 wherein said dosage form further comprises coenzyme Q10 in an amount ranging from about 0.003% to about 2.250% by weight of said dosage form.

6. The method of claim 3 wherein said dosage form further comprises 17 alpha-estradiol in an amount ranging from about 0.008% to about 2.69% by weight of said dosage form.

7. The method of claim 3 wherein said dosage form further comprises glutathione in an amount ranging from about 0.003% to about 2.383% by weight of said dosage form.

8. The method of claim 4 wherein said dosage form further comprises coenzyme Q10 in an amount ranging from about 0.003% to about 2.250% by weight of said dosage form.

9. The method of claim 4 wherein said dosage form further comprises 17 alpha-estradiol in an amount ranging from about 0.008% to about 2.69% by weight of said dosage form.

10. The method of claim 4 wherein said dosage form further comprises glutathione in an amount ranging from about 0.003% to about 2.383% by weight of said dosage form.

11. The method of claim 5 wherein said dosage form further comprises 17 alpha-estradiol in an amount ranging from about 0.008% to about 2.69% by weight of said dosage form.

12. The method of claim 5 wherein said dosage form further comprises glutathione in an amount ranging from about 0.003% to about 2.383% by weight of said dosage form.

13. The method of claim 8 wherein said dosage form further comprises 7 alpha-estradiol in an amount ranging from about 0.008% to about 2.69% by weight of said dosage form.

14. The method of claim 8 wherein said dosage form further comprises glutathione in an amount ranging from about 0.003% to about 2.383% by weight of said dosage form.

15. The method of claim 13 wherein said dosage form further comprises glutathione in an amount ranging from about 0.003% to about 2.383% by weight of said dosage form.

16. A method for treating a patient for the prevention, management and clinical amelioration of chronic glaucoma, ocular aging, periocular aging and conditions giving rise thereto, said method comprising administering to said patient a unit dosage form comprising as an active ingredient a therapeutically effective amount of R-alpha-lipoic acid.

17. The method of claim 16 wherein said active ingredient is formulated as an eyedrop.

18. The method of claim 17 wherein said R-alpha-lipoic acid is in an amount ranging from about 0.026% mg to about 4.00% by weight of said dosage form.

19. The method of claim 18 wherein said dosage form further comprises coenzyme Q10 in an amount ranging from about 0.003% to about 2.250% by weight of said dosage form.

20. The method of claim 18 wherein said dosage form further comprises 17 alpha-estradiol in an amount ranging from about 0.008% to about 2.69% by weight of said dosage form.

21. The method of claim 18 wherein said dosage form further comprises glutathione in an amount ranging from about 0.003% to about 2.383% by weight of said dosage form.

22. The method of claim 19 wherein said dosage form further comprises 17 alpha-estradiol in an amount ranging from about 0.008% to about 2.69% by weight of said dosage form.

23. The method of claim 20 wherein said dosage form further comprises glutathione in an amount ranging from about 0.003% to about 2.383% by weight of said dosage form.

24. The method of claim 22 wherein said dosage form further comprises glutathione in an amount ranging from about 0.003% to about 2.383% by weight of said dosage form.

25. A method for treating a patient for the prevention, management and clinical amelioration of chronic glaucoma, ocular aging, periocular aging and conditions giving rise thereto, said method comprising administering to said patient a unit dosage form comprising as an active ingredient a therapeutically effective amount of coenzyme Q10.

26. The method of claim 25 wherein said active ingredient is formulated as an eyedrop.

27. The method of claim 26 wherein said coenzyme Q10 is in an amount ranging from about 0.003% to about 2.250% by weight of said dosage form.

28. The method of claim 27 wherein said dosage form further comprises 17 alpha-estradiol in an amount ranging from about 0.008% to about 2.69% by weight of said dosage form.

29. The method of claim 27 wherein said dosage form further comprises glutathione in an amount ranging from about 0.003% to about 2.383% by weight of said dosage form.

30. The method of claim 28 wherein said dosage form further comprises glutathione in an amount ranging from about 0.003% to about 2.383% by weight of said dosage form.

31. A method for treating a patient for the prevention, management and clinical amelioration of chronic glaucoma, ocular aging, periocular aging and conditions giving rise thereto, said method comprising administering to said patient a unit dosage form comprising as an active ingredient a therapeutically effective amount of 17 alpha-estradiol.

32. The method of claim 31 wherein said active ingredient is formulated as an eyedrop.

33. The method of claim 26 wherein said 17 alpha-estradiol is in an amount ranging from about 0.008% to about 2.69% by weight of said dosage form.

34. The method of claim 33 wherein said dosage form further comprises glutathione in an amount ranging from about 0.003% to about 2.383% by weight of said dosage form.

35. A method for treating a patient for the prevention, management and clinical amelioration of chronic glaucoma, ocular aging, periocular aging and conditions giving rise thereto, said method comprising administering to said patient a unit dosage form comprising as an active ingredient a therapeutically effective amount of glutathione.

36. The method of claim 35 wherein said dosage form is formulated as an eyedrop.

37. The method of claim 35 wherein said glutathione is in an amount ranging from about 0.003% to about 2.383% by weight of said dosage form.

38. A unit dosage form for the treatment of ocular and periocular disease in which said unit dosage is in the form of an eyedrop, comprising a therapeutically effective amount of tetrahydrobiopterin from about 0.026% to about 4.00% by weight of said dosage form.

39. A unit dosage form for the treatment of ocular and periocular disease in which said unit dosage is in the form of an eyedrop, comprising a therapeutically effective amount of R-alpha-lipoic acid from about 0.002% to about 3.15% by weight of said dosage form.

40. A unit dosage form for the treatment of ocular and periocular disease in which said unit dosage is in the form of an eyedrop, comprising a therapeutically effective amount of coenzyme Q10 from about 0.003% to about 2.250% by weight of said dosage form.

41. A unit dosage form for the treatment of ocular and periocular disease in which said unit dosage is in the form of an eyedrop, comprising a therapeutically effective amount of 17 alpha-estradiol from about 0.008% to about 2.69% by weight of said dosage form.

42. A unit dosage form for the treatment of ocular and periocular disease in which said unit dosage is in the form of an eyedrop, comprising a therapeutically effective amount of glutathione from about 0.003% to about 2.383% by weight of said dosage form.

43. The method of claim 1, wherein said tetrahydrobiopterin is a member selected from the group consisting of tetrahydrobiopterin, L-tetrahydrobiopterin, L-erythro-tetrahydrobiopterin, 6-R-L-erythro-5,6,7,8-tetrahydrohydrobiopterin, 6-1′,2′-dioxypropyl tetrahydropterin, 6-1′-oxo-2′-hydroxypropyl tetrahydropterin, 6-1′-hydroxy-2′-oxypropyl tetrahydropterin, sepiapterin, tetrahydro-sepiapterin, quinonoid 5,6-dihydrobiopterin, and 7,8-dihydrobiopterin.

44. The method of claims 2, 17, 26, 32, 36, 38, 39, 40, 41, or 42 wherein said unit dosage form is a member selected from the group consisting of therapeutically effective formulations consisting of eyedrops, ointments, gels, suspensions, transmembrane delivery systems, electrophoresis systems, periocular injections and other clinically appropriate ocular dosage forms and treatment methods.
